(19) United States
(12) Patent Application Publication
(10) Pub. No.: US 2010/0155600 A1
(43) Pub. Date: Jun. 24, 2010

(54) METHOD AND APPARATUS FOR PLASMA DOSE MEASUREMENT

(75) Inventors: Joseph P Dzengeleski, Newton, NH (US); Timothy J. Miler, Ipswich, MA (US); Jay T. Scheuer, Rowley, MA (US); Christopher J. Leavitt, Gloucester, MA (US)

Correspondence Address: VARIAN SEMICONDUCTOR EQUIPMENT ASSC., INC., 35 DORY RD., GLOUCESTER, MA, 01930-2297, US

(73) Assignee: Varian Semiconductor Equipment Associates, Inc., Gloucester, MA (US)

(21) Appl. No.: 12/342,262

(22) Filed: Dec. 23, 2008

(51) Int. Cl. G01T 1/00; A61N 5/00

(52) U.S. Cl. 250/336.1; 250/492.1

(57) ABSTRACT

An non-Faraday ion dose measurement device is positioned within a plasma process chamber and includes a sensor located above a workpiece within the chamber. The sensor is configured to detect the number of secondary electrons emitted from a surface of the workpiece exposed to a plasma implantation process. The sensor outputs a current signal proportional to the detected secondary electrons. A current circuit subtracts the detected secondary current generated from the sensor and subtracts it from a bias current supplied to the workpiece within the chamber. The difference between the currents provides a measurement of the ion dose current calculated in situ and during the implantation process.

### BACKGROUND OF THE INVENTION

1. Field of the Invention

Embodiments of the invention relate to the field of plasma doping systems. More particularly, the present invention relates to an apparatus and method for measuring the uniformity of a plasma dose implanted into a workpiece or wafer.

2. Discussion of Related Art

Ion implantation is a process used to dope ions into a work piece. One type of ion implantation is used to implant impurity ions during the manufacture of semiconductor substrates to obtain desired electrical device characteristics. An ion implanter generally includes an ion source chamber which generates ions of a particular species using, for example, a series of beam line components to control the ion beam and a platen to secure the wafer that receives the ion beam. These components are housed in a vacuum environment to prevent contamination and dispersion of the ion beam. The beam line components may include a series of electrodes to extract the ions from the source chamber, a mass analyzer configured with a particular magnetic field such that only the ions with a desired mass-to-charge ratio are able to travel through the analyzer, and a corrector magnet to provide a ribbon beam which is directed to a wafer orthogonally with respect to the ion beam to implant the ions into the wafer substrate. The ions lose energy when they collide with electrons and nuclei in the substrate and come to rest at a desired depth within the substrate based on the acceleration energy. The depth of implantation into the substrate is based on the ion implant energy and the mass of the ions generated in the source chamber. Typically, arsenic or phosphorus may be doped to form n-type regions in the substrate and boron, gallium or indium are doped to create p-type regions in the substrate.

Ion implanters as described above are usually associated with relatively high implant energies. When shallow junctions are required in the manufacture of semiconductor devices, lower ion implant energies are necessary to confine the dopant material near the surface of the wafer. In these situations, plasma deposition (PLAD) systems are used where the depth of implantation is related to the voltage applied between the wafer and an anode within a plasma doping chamber. In particular, a wafer is positioned on a platen which functions as a cathode within the chamber. An ionizable gas containing the desired dopant materials is introduced into the plasma chamber. The gas is ionized by any of several methods of plasma generation, including, but not limited to DC glow discharge, capacitively coupled RF, inductively coupled RF, etc. Once the plasma is established, there exists a plasma sheathe between the plasma and all surrounding surfaces, including the workpiece. The platen and workpiece are then biased with a negative voltage in order to cause the ions from the plasma to cross the plasma sheathe and be implanted into the wafer at a depth proportional to the applied bias voltage. A voltage pulse is applied between the platen and an anode (formed by the walls of the plasma chamber) causing formation of a plasma having a plasma sheath in the vicinity of the wafer. The voltage pulse causes the ions in the plasma to cross the plasma sheath and be implanted into the wafer at a desired depth and dose.

Dosimetry is the measurement of the number of ions per unit area implanted in a wafer or workpiece. This is used to determine ion dose levels during the implant process to ensure manufacturing processes and implant recipes. In high energy ion implantations, a Faraday cup is positioned beside or behind a wafer and generates a current proportional to the ion beam as the beam is deflected into the side-mounted Faraday cup or when the wafer is below the ion beam, allowing the beam to enter the rear-mounted Faraday cup. This current is used to determine the ion dose. In shallow or low energy implantations, a Faraday cup is substituted for a wafer to test the ion dose and, once the desired dosage level is achieved, a wafer replaces the Faraday cup. Alternatively, one or more Faraday cups may be positioned adjacent to the platen, as part of the ‘cathode’ and is biased with the platen and wafer for collecting a sample of the positive ions accelerated across the plasma sheath. This sample is representative of the ion dose implanted in the wafer. However, to determine the dose amounts, the Faraday acceptance aperture (the aperture through which the ions pass) must be accurately defined and maintained. During the implant process, this acceptance aperture suffers from erosion and/or deposition as the ions enter the Faraday cup. This causes the aperture area to vary at different rates with time, depending on the chemistry, voltage, and dose levels used, resulting in varying dose calculations which may compromise dose reliability and repeatability. Thus, there is a need to provide a dose measurement device that is used in situ within a plasma chamber during the implantation process which provides accurate plasma implantation dose information associated with a target workpiece or wafer.

### SUMMARY OF THE INVENTION

Exemplary embodiments of the present invention are directed to an ion dose measurement device. In an exemplary embodiment, an ion dose measurement device is includes a power supply connected to a workpiece positioned within a plasma process chamber. The power supply provides a bias current to the workpiece. A sensor is positioned above the workpiece within the chamber and is configured to detect a sample of secondary electrons emitted from a surface of the workpiece which is exposed to a plasma doping. The sensor outputs a current signal proportional to the sample of detected secondary electrons. A current circuit receives the current generated from the sample of secondary electrons and the bias current signal supplied to the workpiece. The current circuit is configured to subtract the current signal generated from the sensor from the bias current signal to determine an ion dose current associated with the workpiece.

### BRIEF DESCRIPTION OF THE DRAWINGS

FIG. 1 is a schematic illustration of a measurement device within a plasma chamber in accordance with an embodiment of the present invention.

FIG. 1A illustrates a schematic view of a current circuit in combination with the plasma chamber shown in FIG. 1

FIG. 2 is a schematic view of a measurement device within a plasma chamber during an exemplary plasma implantation operation in accordance with an embodiment of the present invention.

FIG. 3 is a graph of an exemplary dose measurement calculations in accordance with an embodiment of the present invention.

FIG. 4 is a flow diagram illustrating the steps associated with the dose measurement process in accordance with the present invention.

### DESCRIPTION OF EMBODIMENTS

The present invention will now be described more fully hereinafter with reference to the accompanying drawings, in which preferred embodiments of the invention are shown. This invention, however, may be embodied in many different forms and should not be construed as limited to the embodiments set forth herein. Rather, these embodiments are provided so that this disclosure will be thorough and complete, and will fully convey the scope of the invention to those skilled in the art. In the drawings, like numbers refer to like elements throughout.

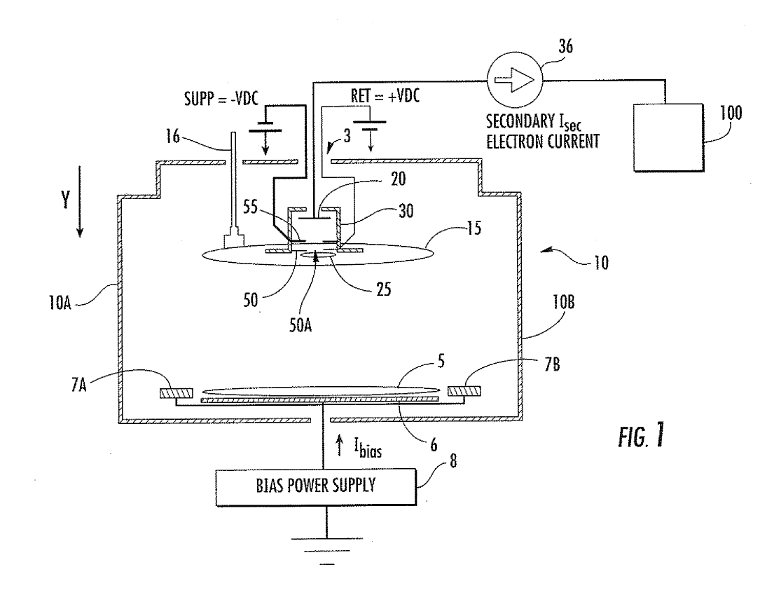

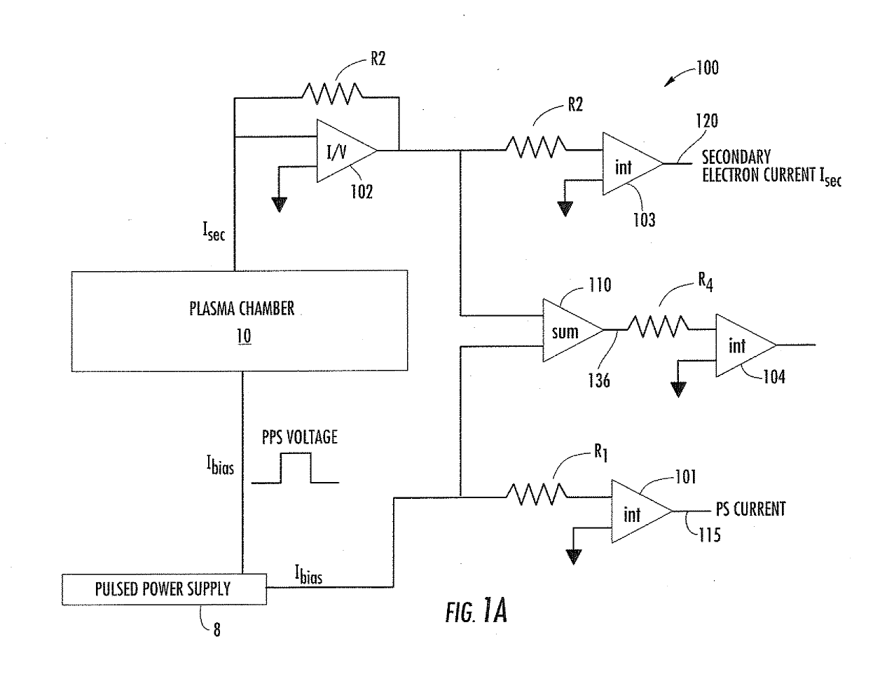

FIG. 1 is a schematic view of the measurement device used in a plasma deposition (PLAD) system. The measurement device includes a sensor 20 mounted above or within a baffle 15 in plasma chamber 10 which is supported above workpiece 5 by support 16. Baffle 15 may be, for example, a gas baffle positioned a distance above a workpiece 5 at one end of the plasma chamber which is configured to facilitate plasma doping for implantation into the workpiece 5. The workpiece may be, for example, a semiconductor wafer mounted on platen 6 which supports the workpiece and provides an electrical connection thereto. A source of ionizable gas (not shown) is introduced into chamber 10 via aperture 3 above the baffle 15 in direction Y at a desired pressure and flow rate. The baffle 15 disperses the gas within the chamber. Although a gas baffle 15 is disclosed, any device positioned above the workpiece 5 which is configured to disburse the gas introduced into the chamber may be employed. The gas is ionized by any of several known techniques. A bias power supply 8 provides a negative voltage pulse to the platen 6, workpiece 5 with respect to an anode formed by the baffle 15, and the walls 10A and 10B of chamber 10. The negative voltage applied to platen 6 which is thereby applied to workpiece 5 attracts the plasma ions across a plasma sheath. The voltage pulses accelerate the ions within the plasma which implant into workpiece 5 as an ion dose to form areas of impurity dopants within the workpiece. The voltage pulses correspond to the implantation depth of the ions into the workpiece which may also be influenced by the pressure and flow rate of the gas introduced into chamber 10, position of the anode as well as the duration of the pulses. The ion dose is the amount of ions implanted into workpiece 5 or the integral over time of the ion current. A pair of Faraday cups 7A and 713 may also be positioned on either side of platen 6 to verify dose levels detected by the measurement device of the present invention during plasma recipe set-up. In this manner, a comparison of these measurements detected by the Faraday cups and the sensors may be performed during recipe development. In addition, a calibration can be determined between the two measurements, and this calibration can be monitored over time to aide in detection of deposition on, or erosion of, the Faraday aperture as discussed above. The change in the relationship of these two measurements may be used to indicate a need for Faraday cup maintenance.

The baffle 15 includes aperture 25 positioned radially along the surface of the baffle. Cavity 30 is aligned with aperture 25 within which sensor 20 is housed. Cavity 30 shown in FIG. 1 is exaggerated for ease of explanation and would typically correspond with the cross sectional thickness of baffle 15. Although the present description of the sensor 20 is disclosed as being integrally formed with baffle 15, the sensor may be housed separately and mounted to baffle 15 or positioned above workpiece 5 separately from baffle 15. In addition, the sensor 20 and associated cavity 30 may be integrally formed with a chamber liner disposed within chamber 10 as long as the sensor is positioned above workpiece 5. Low voltage electrostatic grids 50 and 55 are configured in front of the detector 20 to discriminate between relatively high energy secondary electrons and low energy plasma ions and electrons. In particular, a first grid 25 is configured nearest the plasma to prevent the low voltage grids from effecting the plasma in the vicinity of the detector. A second grid 50 is disposed between sensor 20 and workpiece 5 and extends a distance across grid 25. Grid 50 includes a screen portion 50A to allow secondary electrons to pass through the aperture to sensor 20. Because aperture 25 is not biased, it does not suffer from unwanted deposition or erosion from the secondary electrons or the low energy plasma ions and electrons passing through the aperture 25. This is distinguished from a Faraday cup aperture which does suffer from unwanted deposit build-up and erosion during the implantation process. Grid 50 is biased with a positive DC voltage (+VDC) and is configured to prevent low energy ions from the plasma within chamber 10 from leaking to sensor 20 during implantation. A second grid 55 is disposed between sensor 20 and first grid 50 and extends across aperture 25 similar to grid 50. Grid 55 includes a corresponding screen portion 55 to allow secondary electrons to pass through the aperture 25 to sensor 20, Grid 55 is biased with a negative DC voltage (−VDC). This negative voltage is substantially below the energy of the implant generated secondary electrons. Thus, when secondary electrons pass through aperture 25 within cavity 30, the negatively charged secondary electrons lose too much energy and become trapped within the cavity, absorbed by sensor 20 and counted via generation of current signal 36. In addition, because second grid 55 is negatively biased, plasma electrons that enter cavity 30 are repelled back toward workpiece 5 by second grid 55. Alternatively, sensor 20 may be positively biased which would obviate the need for second grid 55. Although sensor 20 is illustrated in FIG. 1 as being centrally disposed above workpiece 5, the location of the sensor may be defined by the user and positioned radially about baffle 15. Aperture 25 is dimensioned to allow absolute calculation of dose by the ratio of this collection area to the entire collection area determined by area of the cathode defined by the workpiece 6 and annular Faraday areas.

As will be described in more detail below, sensor 20 detects the number of secondary electrons which pass through aperture 26 and generates a current signal 36 proportional to the number of secondary electrons detected. These secondary electrons are generated from the surface of workpiece 5 when plasma ions are implanted into workpiece 5. The current signal 36 is returned to ground and is used to determine the ion dose implanted into workpiece 5. Alternatively, a resistor may be disposed between sensor 20 and ground potential. The ion dose measurement method of the present invention utilizes Kirchoffs Current Law (KCL) where the algebraic sum of all currents entering and leaving a circuit node is equal to zero. Workpiece 5 is the node and the current entering the workpiece is I_{bias} supplied by bias power supply 8. The current leaving the node or workpiece is I_{sec} which is the current generated by the detection of secondary electrons by sensor 20. Thus, the ion current entering the workpiece or wafer (I_{wafer}) is the difference between the bias current from power supply 8 and the current 36 generated by the secondary electrons and detected by sensor 20. Accordingly, by applying KCL to these currents the ion current supplied to the workpiece 5 is determined by I_{wafer}=I_{bias}−I_{sec} Secondary electron current 36 may be supplied to a current circuit 100 or integrator to determine the ion dose current implanted into the workpiece. This method of determining ion dose, which requires accurate measurement of secondary electrons emitted from the workpiece, eliminates the need to have prior knowledge of the secondary emission coefficient of the workpiece surface.

FIG. 1A illustrates a schematic view of circuit 100 in combination with plasma chamber 10 shown in FIG. 1 in an exemplary embodiment of the present invention. In particular, the power supply 8 may be a pulsed power supply providing current I_{bias} to platen 6. The bias current I_{bias} is also supplied to current circuit 110. The bias current I^{bias} is supplied to output 115 via resistor R1 and integrator circuit 101. The secondary current I_{sec} generated from sensor 20 is supplied to current circuit 110 via inverter 102. A feedback loop having resistor R2 may also be employed. The secondary electron current I_{sec} is supplied to output 120 via resistor R2 and integrator circuit 103. Current circuit 110 may be a current summation circuit configured to receive the secondary electron current I_{sec} and the bias current I_{bias} output current 136 consistent with KCL to determine I_{wafer} where I_{wafer}=I_{bias}−I_{sec}. An integrator circuit 104 and resistor R4 may also be connected to circuit 110.

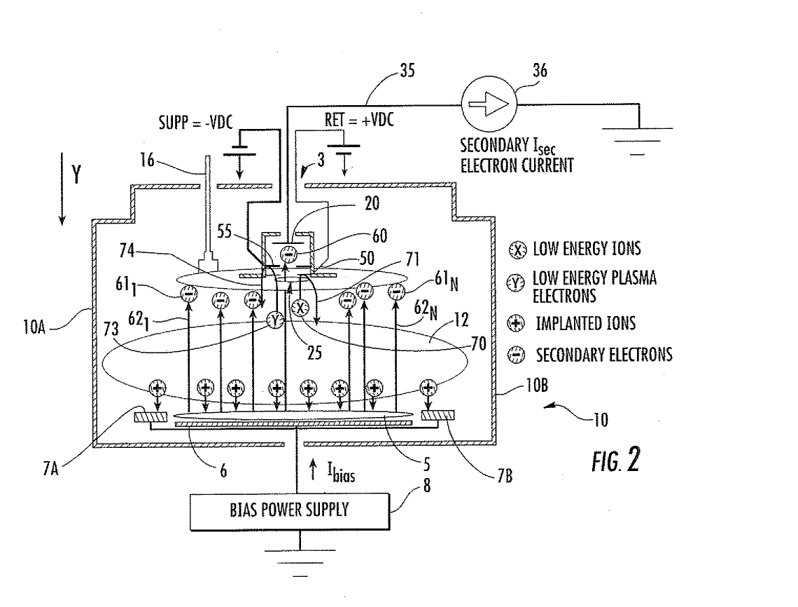

FIG. 2 is a schematic view of the measurement device during a plasma implantation operation. In particular, an ionizable gas is introduced into chamber 10 above baffle 15 via aperture 3 in direction Y at a desired pressure and flow rate. Plasma 12 is then created in the plasma chamber 10 by addition of energy by any of the known methods. Bias power supply 8 provides a negative voltage bias to workpiece 5 with respect to the anode formed by the walls of chamber 10 and the gas baffle 15. This causes the formation of a plasma containing positive ions (depicted with a “+” sign in FIG. 2) which are accelerated through plasma sheath 12 and implanted into workpiece 5 to form areas of impurity dopants. When the ions are implanted into workpiece 5, secondary electrons (depicted with a “−” sign in FIG. 2) are emitted from the surface of workpiece 5 which are then accelerated orthogonally and are reflected upward toward sensor 20. The energy of the secondary electrons is determined by the implant voltage of the plasma ions as the electrons are accelerated across the plasma sheath 12 above workpiece 5. This energy allows the secondary electrons to be discriminated from low energy plasma electrons by means of the low voltage electrostatic grids 50 and 55 disposed between sensor 20 and workpiece 5. These secondary electrons are detected by sensor 20 and a proportional current signal 36 is generated and subtracted from the bias current (I_{bias}) supplied by power supply 8 to determine the dose current I_{wafer}. For example, secondary electrons 60 are emitted from the surface of workpiece 5 orthogonally. Those secondary electrons 60 aligned with cavity 30 pass through aperture 25 to low voltage first grid 50 and low voltage second grid 55. The secondary electrons are received and counted by sensor 20. Because aperture 26 is not biased and it is protected from plasma by grids 50 and 55, aperture 26 is not effected by plasma chemistry within chamber 10 and does not suffer from deposition or erosion thereof. Additionally, a ground reference grid (not shown) may be connected to the first grid 50 and second grid 55 and disposed between the first grid 50 and the workpiece. This reference grid is configured to clamp the electric fields associated with the first and second grids 50 and 55 and prevent them from effecting the plasma in the vicinity of sensor 20.

In response to the detection of secondary electrons 60, sensor 20 generates current 36 via line 35. This current 36 indicates the number of secondary electrons emitted from the surface of workpiece 5. As noted earlier, this current 36 may be supplied to a current circuit, integrator or other standard current detection device which subtracts current 36 from the bias current I_{bias} supplied by power supply 8 and outputs a current value consistent with KCL. In this manner, the current circuit provides the ion dose current implanted in workpiece 5 by determining I_{wafer}=I_{bias}−I_{sec}.

Secondary electrons 61₁-61_{N} which are emitted orthogonally from the surface of workpiece 5 as indicated by arrows 62₁-62_{N} are not aligned with cavity 30 and thus, are not detected by sensor 20. Low energy plasma ions 70 (depicted with an “X” in FIG. 2) do not have enough energy to be implanted into workpiece 5 and reflect orthogonally upward toward baffle 15. The low energy plasma ions 70 enter cavity 30 via aperture 25. Because grid 50 is biased with a positive DC voltage (+VDC), this low energy plasma ion which also has a positive charge is prevented from further entering cavity 30 toward sensor 20. Low energy plasma ion 70 is repelled back toward workpiece 5 as indicated by arrow 71. Plasma electron 73 (depicted with a “Y” in FIG. 2) may also enter cavity 30. This representative plasma electron passes through aperture 25 and because second grid 55 is biased with a low negative DC voltage (−VDS), plasma electron 73 is repelled back toward workpiece 5 as indicated by arrow 74 and is not counted by sensor 20. In this manner, the measurement device detects only the secondary electrons emitted from the surface of workpiece 5 in situ and during ion implantation to monitor the ion dose of the particular plasma.

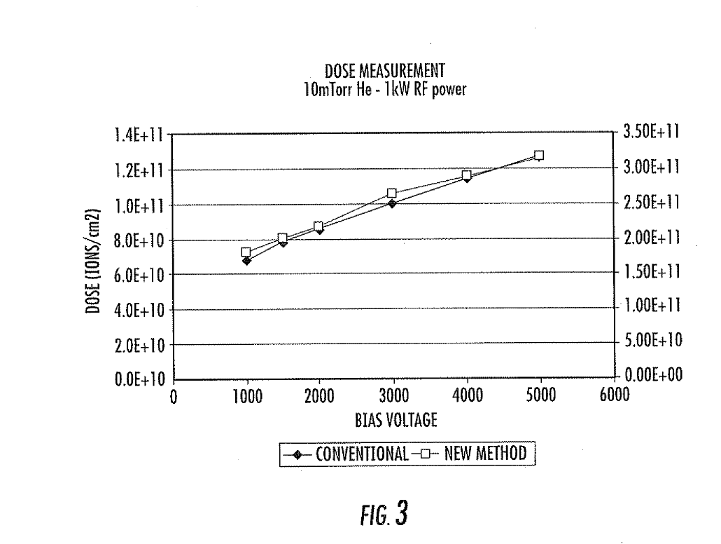

FIG. 3 is a graph of an exemplary dose measurement utilizing the present invention as compared to a conventional measurement utilizing a Faraday cup. The exemplary dose measurement was' detected for helium at 10 mTorr and 1 kW RF power within an ion source chamber. The ion dose measurements were detected using both methods at the various bias voltages. Again, the bias voltage is used to accelerate the ions within the plasma and attract them across the plasma sheath toward the workpiece for implantation. As can be seen, the dose measurement utilizing the conventional Faraday cup method (labeled “conventional”) and the method of the present invention (labeled “new method”) have the same characteristic slope. In this manner, ion dose measurements can be detected in-line during the implantation process without concerns related to Faraday aperture precision problems. In practice, a short duration spike of displacement current may appear at the beginning and end of each wafer biasing pulse. The magnitude of this displacement current is proportional to the capacitance of the system. In a measurement scheme that integrates all current during each pulse, this displacement current integrates to zero. In embodiments which take discrete samples of I_{bias} and I_{sec} during each pulse, the effect is mitigated by sampling near the mid-point in time of the pulse.

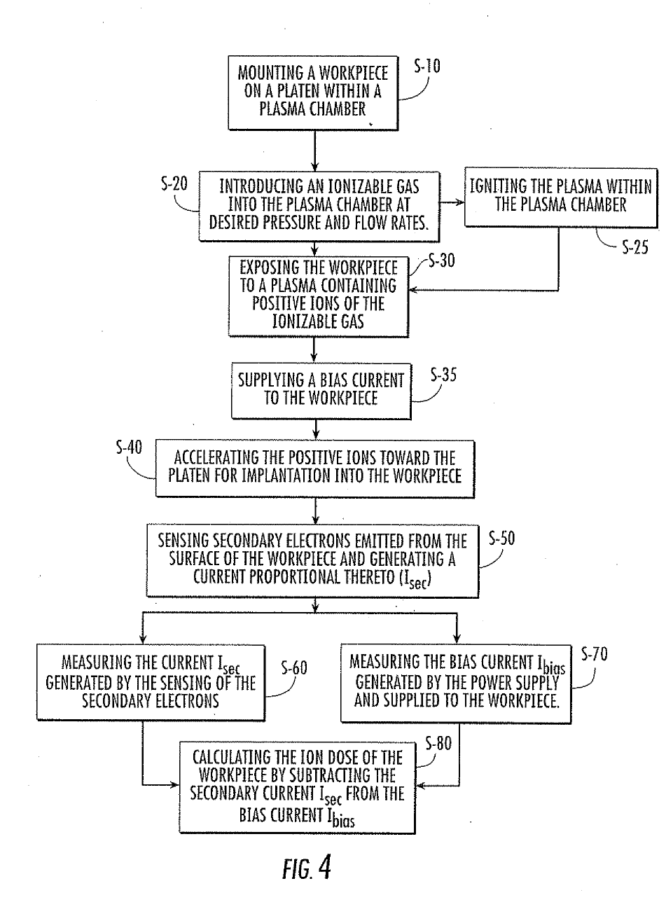

FIG. 4 is a flow diagram illustrating the steps associated with the dose measurement process in accordance with the present invention. A workpiece 5 is mounted on a platen or support within a plasma chamber 10 at step S-10. An ionizable gas is introduced into the plasma chamber at step S-20 and the plasma is ignited at step S-25. The workpiece 5 is exposed to a plasma containing positive ions contained in the ionizable gas at step S-30. The workpiece 5 is biased with a current I_{bias} supplied by power supply 8 at step S-35. The positive ions are accelerated to an implant energy toward the platen for implantation into the workpiece 5 at step S-40. At step S-50, secondary electrons which are emitted from the surface of the workpiece are detected. A current I_{sec} which is proportional to the number of secondary electrons detected is generated by sensor 20. This current I_{sec} is measured at step S-60. The bias current I_{bias} which is supplied to the workpiece at step S-25 is measured at step S-70. At step S-80, the ion does of the workpiece is determined by subtracting the secondary electron current I_{sec} measured at step S-60 from the bias current I_{bias} measured at step S-70.

While the present invention has been disclosed with reference to certain embodiments, numerous modifications, alterations and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An ion dose measurement device within a plasma process chamber, said measurement device comprising:
a power supply connected to a workpiece positioned within said plasma process chamber, said power supply providing a bias current to said workpiece;
a sensor positioned above said workpiece within said chamber, said sensor configured to detect a sample of secondary electrons emitted from a surface of said workpiece exposed to a plasma doping and output a current signal proportional to said sample of detected secondary electrons; and
a current circuit configured to receive said current generated from said sample of secondary electrons and said bias current signal supplied to said workpiece, said current circuit configured to subtract said current signal generated from said sensor from said bias current signal to determine an ion dose current associated with said workpiece.

2. The ion dose measurement device of claim 1 further comprising a housing having a cavity within which said sensor is mounted, said cavity defining an aperture through which said secondary electrons pass, said housing positioned above said workpiece within said chamber.

3. The ion dose measurement device of claim 2 wherein said device housing is mounted on a baffle within said process chamber.

4. The ion dose measurement device of claim 1 wherein said sensor is integrally formed in a baffle within said process chamber.

5. The ion dose measurement device of claim 1 wherein said sensor is integrally formed with a liner of said plasma process chamber.

6. The ion dose measurement device of claim 1 further comprising a grid disposed between said sensor and said workpiece, said grid biased with a positive DC voltage and configured to prevent low energy ions from said plasma doping from contacting said sensor.

7. The ion dose measurement device of claim 6 wherein said grid is a first grid, said measurement device further comprising a second grid disposed between said first grid and said sensor, said second grid biased with a negative DC voltage and configured to trap said secondary electrons toward said sensor.

8. The ion dose measurement device of claim 7 further comprising a referenced grid connected to said first and second grids, said reference grid configured to clamp electric fields associated with said first and second grids.

9. A plasma doping system comprising:
a plasma doping chamber configured to receive an ionizable gas;
a platen mounted in said plasma doping chamber for supporting a workpiece;
a power supply connected to said workpiece and configured to supply a bias current to said workpiece
a source of ionizable gas coupled to said chamber, said ionizable gas containing a desired dopant for implantation into said workpiece;
a plasma source for producing a plasma containing positive ions of said ionizable gas, and accelerating said positive ions toward said platen for implantation into said workpiece;
a sensor disposed above said workpiece within said plasma doping chamber, said sensor configured to detect the number of secondary electrons emitted from said positive ions of said plasma hitting said surface of said workpiece, said sensor configured to output a current signal proportional to said number of detected secondary electrons; and
a current circuit configured to receive said current generated from said sample of secondary electrons and said bias current signal supplied to said workpiece, said current circuit configured to subtract said current signal generated from said sensor from said bias current signal to determine an ion dose current associated with said workpiece

10. The plasma doping system of claim 9 further comprising a housing having a cavity within which said sensor is mounted, said cavity defining an aperture through which said secondary electrons pass, said housing positioned above said workpiece within said chamber.

11. The plasma doping system of claim 10 wherein said device housing is mounted on a baffle within said plasma doping chamber.

12. The plasma doping system of claim 9 wherein said sensor is integrally formed in a baffle within said plasma doping chamber.

13. The ion dose measurement device of claim 8 wherein said sensor is integrally formed with a liner of said plasma process chamber.

14. The plasma doping system of claim 9 further comprising a grid disposed between said sensor and said workpiece, said grid biased with a positive DC voltage and configured to prevent low energy ions passing through said aperture toward said sensor.

15. The plasma doping system of claim 14 wherein said grid is a first grid, said plasma doping system further comprising a second grid disposed between said first grid and said sensor, said second grid biased with a negative DC voltage and configured to trap said secondary electrons within said cavity.

16. A method of measuring plasma implant dose current comprising:
mounting a workpiece on a platen within a plasma chamber;
introducing an ionizable gas into said plasma chamber;
exposing said workpiece to a plasma containing positive ions of said ionizable gas;
applying a bias current to said workpiece;
accelerating said positive ions to an implant energy through a plasma sheath;
directing said accelerated ions toward said platen for implantation into said workpiece; and
sensing secondary electrons emitted from a surface of said workpiece when said plasma ions are implanted into said workpiece;
generating a current proportional to the number of secondary electrons sensed;
measuring the current generated by the sensed secondary electrons;
measuring the bias current supplied to the workpiece;
subtracting the current generated by the sensed secondary electrons from the bias current supplied to the workpiece.
